(19) United States
(12) Patent Application Publication
(10) Pub. No.: US 2012/0239085 A1
(43) Pub. Date: Sep. 20, 2012

(54) SUTURE IMPLANT COMPONENT AND SUTURE IMPLANT MECHANISM INCLUDING SUCH A COMPONENT

(76) Inventors: Ryan SCHLOTTERBACK, Fort Wayne, IN (US); Dale R. PETERSON, La Jolla, CA (US); Kevin L. OHASHI, Jamaica Plain, MA (US)

(21) Appl. No.: 13/232,114

(22) Filed: Sep. 14, 2011

Related U.S. Application Data
(60) Provisional application No. 61/383,665, filed on Sep. 16, 2010.

(30) Foreign Application Priority Data
Sep. 23, 2010 (FR) 1057677

(51) Int. Cl. A61B 17/04

(52) U.S. Cl. 606/228; 606/232

(57) ABSTRACT

A system for suture implantation according to embodiments of the present invention includes a suture implant component including a body which extends along a longitudinal dimension, the body including an opening configured to receive a suture, the body configured for attachment to the suture, the body being deformable, upon application of a tension to the suture, between a resting configuration in which the body has a first longitudinal dimension and a first transverse dimension, and an active configuration in which the body has a second longitudinal dimension smaller than the first longitudinal dimension and a second transverse dimension larger than the first transverse dimension, and further including a wall arranged about the longitudinal dimension, wherein the wall is deformable in an accordion-like manner as the body deforms from the resting configuration to the active configuration.

### CROSS-REFERENCE TO RELATED APPLICATION

This application claims the benefit of U.S. Provisional Patent Application Ser. No. 61/383,665, filed Sep. 16, 2010, and claims foreign priority to French Patent Application No. FR1057677, filed on Sep. 23, 2010, both of which are incorporated by reference herein in their entireties for all purposes.

### TECHNICAL FIELD

Embodiments of the present invention relate generally to suture implants for fixation of a repaired soft tissue to a bone, such as a muscle, a tendon, or a ligament.

### BACKGROUND

In a known manner, surgeons use different mechanisms for reattaching soft tissue to a bone to promote healing, including, for example, screws, staples, pins, nails, or even a single suture. A bone cavity may be prepared in a bone of a patient at the point at which re-attachment of the soft tissue is desired, and a suture implant may be anchored in the cavity to permit fixation of a surgical suture. The implant may be threaded, inserted by force into the cavity, or locked in place after insertion.

However, the known mechanisms for repairing the glenoidal labrum and other soft tissues present positioning challenges, as well as certain risks: bone loss or osteoporosis, reaction with polymers, rupture of certain elements, and elements which remain in the patient's body and interfere with healing. In addition, the retreat of bone may be increased by the material which is implanted. Also, such surgical repairs may benefit from being minimally invasive, in other words leaving the most material possible in the body, while presenting a significant tissue-to-bone interface.

### SUMMARY

A system for suture implantation according to embodiments of the present invention includes a suture implant component, which includes a body which extends along a longitudinal dimension, the body including an opening configured to receive a suture. The body may be configured for attachment to the suture, and may be deformable, upon application of a tension to the suture, between a resting configuration in which the body has a first longitudinal dimension and a first transverse dimension, and an active configuration in which the body has a second longitudinal dimension smaller than the first longitudinal dimension and a second transverse dimension larger than the first transverse dimension. Such a system may further include a wall arranged about the longitudinal dimension, wherein the wall is deformable in an accordion-like manner as the body deforms from the resting configuration to the active configuration.

The system may further include the suture, which cooperates with the body to form a general U or V shape, and wherein the suture is attached to the body at at least a distal end of the body. The wall is deformable in the accordion-like manner by pleating, bending, crushing, or contraction as the body deforms from the resting configuration to the active configuration, according to embodiments of the present invention. The wall may be tubular or conical. The body is formed about a longitudinal axis extending along the longitudinal dimension, and the wall is arranged about the longitudinal axis, such that in the active configuration, the wall forms several folds about the longitudinal axis, according to embodiments of the present invention.

According to embodiments of the present invention, the body is woven or braided with biocompatible fibers formed with one or a combination of materials selected from the group consisting of: polyester, polyethylene, and polyhydroxyalkanoate. The biocompatible fibers may be resorbable fibers. According to some embodiments, the suture implant component includes at least two types of fibers each having a different diameter. In some cases, the suture includes a first strand and a second strand, wherein the first and second strands traverse the distal end of the body and are connected by an arc, wherein the arc comprises a knot which is located outside of the distal end of the body.

The outside wall of the body may include monofilaments distributed along at least a portion of the longitudinal dimension. The body may further include a reinforcing ring located at a proximal end of the body. According to embodiments of the present invention, the proximal end of the body includes an opening while a distal end of the body is closed. A locking element, for example a clip, staple, ring, and/or button may be disposed in the body to maintain the body in the active configuration, according to embodiments of the present invention. According to embodiments of the present invention, the body further includes a cinch mechanism at its distal end, wherein the cinch mechanism is configured to receive and lock the suture, the cinch mechanism formed of the same material as the suture and woven into the body. The system may further include a means for attaching the suture to the distal end of the body.

According to embodiments of the present invention, the system may further include an insertion rod and a tubular sleeve. The rod may be adapted to maintain the first longitudinal dimension and the first transverse dimension of the body in the resting configuration, the rod configured to extend through the opening and to occupy an interior space of the body, according to embodiments of the present invention. The sleeve may be tubular and adapted to maintain a proximal end of the body in position during deformation of the body from the resting configuration to the active configuration by pressing against the proximal end of the body, according to embodiments of the present invention. In some cases, the rod includes a point configured to punch or pierce a hole into bone for insertion of the suture implant component into the bone.

A method for implanting a suture implant component according to embodiments of the present invention, wherein the suture implant component includes a body which extends along a longitudinal axis, the body including an opening configured to receive a suture, the body configured for attachment to the suture, the body further including a wall arranged about the longitudinal axis, includes: inserting a rod into the body, inserting a sleeve over the rod, into sliding engagement with the rod, inserting the body and a distal end of the rod into a bone cavity, removing the rod from the body, and maintaining the sleeve in place against a proximal end of the body as a tension force is applied to the suture to deform the body, in an accordion-like manner, from a resting configuration in which the body has a first longitudinal dimension and a first transverse dimension, to an active configuration in which the body has a second longitudinal dimension smaller than the first longitudinal dimension and a second transverse dimension larger than the first transverse dimension, thereby securing the body within the bone cavity.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 illustrates a perspective view of a suture implant mechanism including a suture implant component, according to embodiments of the present invention.

FIG. 2 illustrates an enlarged perspective view of the suture implant mechanism of FIG. 1 taken along circle II of FIG. 1, according to embodiments of the present invention.

FIG. 3 illustrates a partial front cross-sectional view of a suture implant mechanism and suture implant component in a bone aperture, according to embodiments of the present invention.

FIG. 4 illustrates the partial front cross-sectional view of FIG. 3 with the insertion rod withdrawn, according to embodiments of the present invention.

FIG. 5 illustrates the partial front cross-sectional view of the suture implant mechanism of FIGS. 3 and 4, with the suture implant component in an active configuration.

FIG. 6 illustrates a front cross-sectional view of another suture implant mechanism in a resting configuration, according to embodiments of the present invention.

FIG. 7 illustrates a front cross-sectional view of the suture implant mechanism of FIG. 6 in an active configuration, according to embodiments of the present invention.

FIG. 8 illustrates a front cross-sectional view of yet another suture implant mechanism in a resting configuration, according to embodiments of the present invention.

FIG. 9 illustrates a front cross-sectional view of the suture implant mechanism of FIG. 8 in an active configuration, according to embodiments of the present invention.

FIG. 10 illustrates a front cross-sectional view of yet another suture implant mechanism in a resting configuration, according to embodiments of the present invention.

FIG. 11 illustrates a front cross-sectional view of yet another suture implant mechanism in a resting configuration, according to embodiments of the present invention.

FIG. 12 illustrates a front cross-sectional view of yet another suture implant mechanism in a resting configuration, according to embodiments of the present invention.

FIG. 13 illustrates a front cross-sectional view of yet another suture implant mechanism in a resting configuration, according to embodiments of the present invention.

FIG. 14 illustrates a front cross-sectional view of the suture implant mechanism of FIG. 13 in a partially active configuration, according to embodiments of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

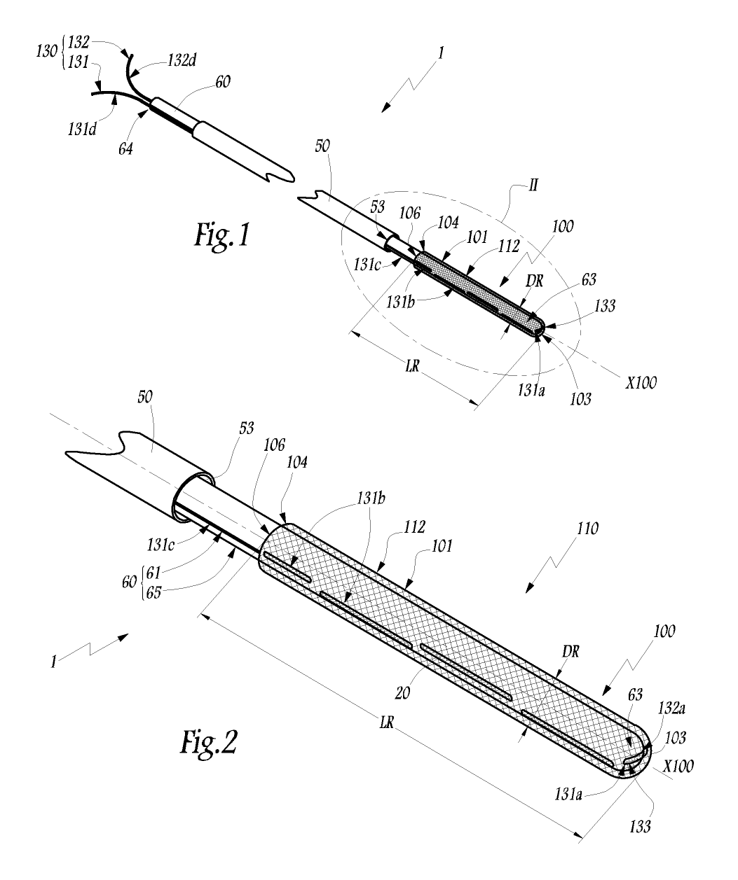

FIGS. 1 to 5 illustrate a suture implant mechanism 1, which includes a suture implant component 100. The mechanism 1 includes a suture 130, a compression sleeve 50 and an insertion rod 60 adapted to cooperate with a body 101 of the component 100, according to embodiments of the present invention.

The sleeve 50 and rod 60 constitute a removable insertion mechanism of the suture implant component 100 in a bone cavity 81, prepared for that purpose by the surgeon in the bone 80 of the patient. The rod 60 has a distal end 63, a proximal end 64, a middle section 65, as well as two grooves 63 and 64. The sleeve 50 may be tubular and may include an annular distal opening 53. In practice, the rod 60 is inserted in a sliding manner in the sleeve 50, according to embodiments of the present invention.

The body 101 of the component 100 may be formed as an elongated tube, with a deformable wall 112 which extends along a central axis X100 and forms an interior space 105, according to embodiments of the present invention. The wall 112 is arranged around the central axis X100, according to embodiments of the present invention. The body 101 has a closed distal end 103, as well as a proximal end 104 provided with an opening 106, according to embodiments of the present invention.

The body 101 of the component 100 may be fabricated by weaving or braiding, or by any other appropriate technique, according to embodiments of the present invention. The sutures or bundles of suture, fibers, or strands are interlaced to form the body 101, according to embodiments of the present invention. The fibers utilized may be identical, or different types of fibers may be used to provide variable materials or dimensions. For example, the body 101 may have at least two types of fibers having different respective diameters. According to some embodiments of the present invention, certain parts or sections of the body 101 may include threading or be reinforced before changing the flexibility of the body 101, so as, for example, to favor deformation of the body 101 according to a particular geometry.

The body 101 and the suture 130 may be made of a resorbable material, for example polyhydroxyalkanoate, in particular poly-4-hydroxybutyrate, such as TephaFLEX™, according to embodiments of the present invention. According to some embodiments of the present invention, the body 101 and the suture 130 are minimally invasive and include mechanical resistance properties conducive to the repair of soft tissue.

One or more materials may be used for the one or more different types of fibers constituting the body 101 of the component 100. The suture may be made of polyester, polyethylene, and/or polyhydroxyalkanoate, according to embodiments of the present invention.

The suture 130 may be implanted into the component 100 during the surgical operation. The suture 130 may include two strands 131 and 132 which are substantially rectilinear in their position of use. The strands 131 and 132 may be joined by an arc 133 disposed at the distal end 103 of the body 101, according to embodiments of the present invention. More precisely, the strand 131 includes several longitudinal portions 131a, 131b, 131c, and 131d, each having a particular function and position. The strand 132 may also include several longitudinal portions 132a, 132b, 132c, and 132d, which may be similar to corresponding portions of stand 131, according to embodiments of the present invention.

At portion 131a, the strand 131 passes through the distal end 103 of the body 101 to rejoin the arc 133. At portion 131b, which extends between the distal end 103 and the proximal end 104 of the body 101, the strand 131 is interlaced with the body 101, as shown in FIGS. 1 and 2, according to embodiments of the present invention. At portion 131c, the strand 131 is lodged in the groove 61 of the rod 60 so as to maintain its position during insertion of the component 100 into the cavity 81, according to embodiments of the present invention. Finally, the portion 131d emerges from the groove 61 at the proximal end 64 of the rod 60, according to embodiments of the present invention.

First, the suture 130 is implanted along with the component 100 with their insertion in the bone cavity 81. Due to the interlacing of the strands 131 and 132 and the arc 133, the suture 130 cooperates mechanically with the body 101. In other words, the portions 131a and 131b of strand 131, the portions 132a and 132b of the strand 132, and the arc 133 form a mechanism of cooperation between the suture 130 and the body 101 of the component 100, according to embodiments of the present invention.

The rod 60 is inserted into the component 100 through the opening 106 which is situated at the proximal end 104. Also, the distal end 63 of the rod 60 is positioned at the level of the distal end 103 in the interior space 105 of the body 101. The two strands 131, 132 are lodged in the grooves 61 and 62, according to embodiments of the present invention. The sleeve 50 is positioned on the rod 60 in sliding engagement in the middle section 65, according to embodiments of the present invention. The sleeve 50 and the rod 60 are arranged coaxially in the component 100, according to embodiments of the present invention.

At this stage, the component 100 is located in a resting configuration. The body 101 has a resting longitudinal dimension LR and a resting transverse dimension DR, such that, with respect to axis X100, the longitudinal dimension corresponds to the length of the body 101, and the transverse dimension corresponds to the diameter of the body 101.

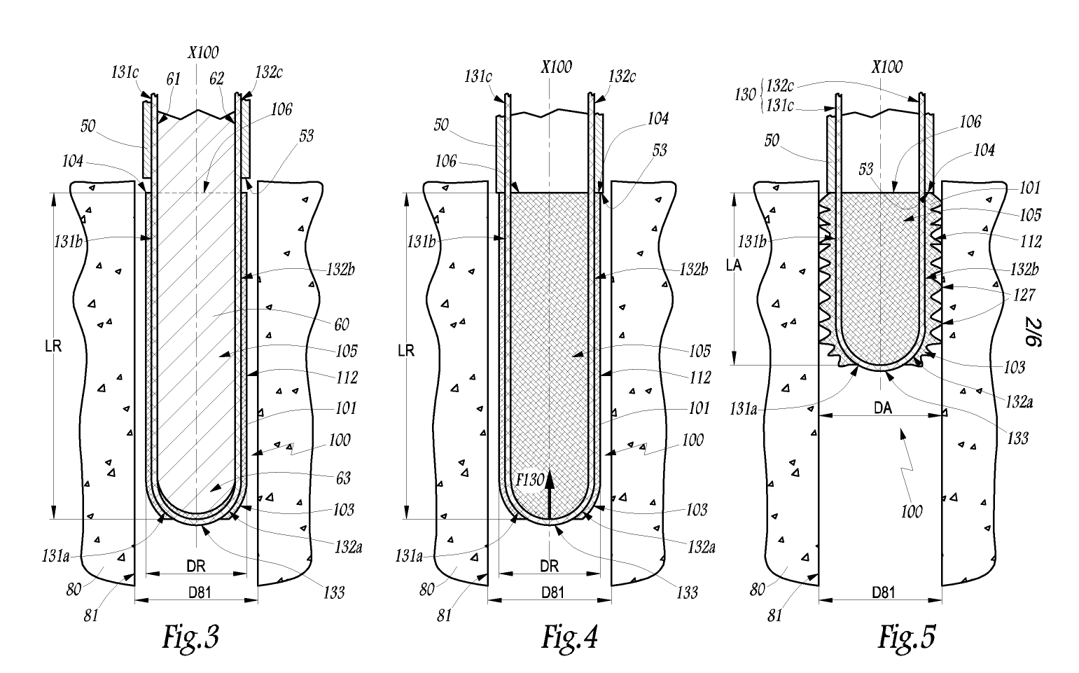

As shown in FIG. 3, the surgeon manipulates the mechanism 1, and more particularly the rod 60, to insert the component 100 in its resting configuration in the bone cavity 81. The diameter DR of the body 101 is smaller than the diameter D81 of the cavity 81. Due to the presence of the rod 60, in the resting configuration, the body 101 does not deform. In other words, the bar 60 permits maintenance of the stability of the longitudinal dimension LR and the transverse dimension DR of the body 101.

As illustrated in FIG. 4, the rod 60 is then withdrawn from the mechanism 1. The rod 60 slides against the component 100 then the sleeve 50, and the strands 131 and 132 slide out of the grooves 61 and 62. At this stage, the mechanism 1 may be manipulated due to the sleeve 50 and the portions 131d and 132d of the suture 130, according to embodiments of the present invention.

In practice, a tension F130 is then exerted on the suture 130. As the suture 130 cooperates with the body 101, and in particular with its end 103, the tension F130 tends to extract the component 100 out of the cavity 81. However, the sleeve 50 is adapted to maintain the proximal end 104 in position as the body 101 is deformed. In other words, the proximal end 104 of the body 101 comes into contact against the distal end 53 of the sleeve 50.

Also, as illustrated in FIG. 5, the body 101 of the component 100 is compressed against the sleeve 50 under the effect of the tension F130. The body 101 is deformed between the resting configuration and the active configuration, in which the body 101 has an active longitudinal dimension LA of a value which is smaller than the value of the resting longitudinal dimension LR, and an active transverse dimension DA of a value which is larger than the value of the resting transverse dimension DR. The initially tubular wall 112 deforms, such that in its active configuration, the deformed wall 112 substantially locks with pressure against the walls of the bone cavity 81, according to embodiments of the present invention. This lodges the wall 112 within the bone cavity 81, according to embodiments of the present invention In particular, the deformation in an “accordion” fashion of the wall 112 about the axis X100 forms several folds. The cooperation between the portions 131b and 132b of suture 130 and the wall 112 permits the body 101 to deform in a controlled manner. Certain strands or fibers woven or braided constituting the body 101 are deformed so as to project from the wall 112, forming some bumps or interferences 127 which hinder the relative movement between the cavity 81 and the component 10. In other words, the deformed wall 112 has several interferences 127 which serve to anchor the component 100 in the boney cavity 81, with an adherence by mechanical cooperation through pinching, friction, and/or seizing.

At this stage, the body 101 is “contracted” in the longitudinal direction LA and “dilated” in the transverse direction DA. The interferences 127, generated by the controlled pleating and/or the bending of the body 101 in accordion-like fashion in the cavity 81, permit the component 100 to resist the tension F130 away from the cavity 81. As such, the component 100 and the suture 130 are rigidly maintained in position in the cavity 81 during the soft tissue repair, according to embodiments of the present invention.

Moreover, the weaving or the braiding of the body 101 may be optimized to favor the formation of interferences 127 and to improve the adherence in the bone cavity 81. For example, in one embodiment, strands or fibers of various sizes may be used in the body 101. According to another example, a larger strand may be twisted around the wall 122 in its resting configuration, similar to an exterior threading. The interferences 127 may be radial, regularly spaced on the wall 112, or wrapped in spiral on the wall 112. As such, the fixation of the component 100 in its active configuration, by adherence of the wall 112 in the cavity 81, is facilitated and reinforced, according to embodiments of the present invention.

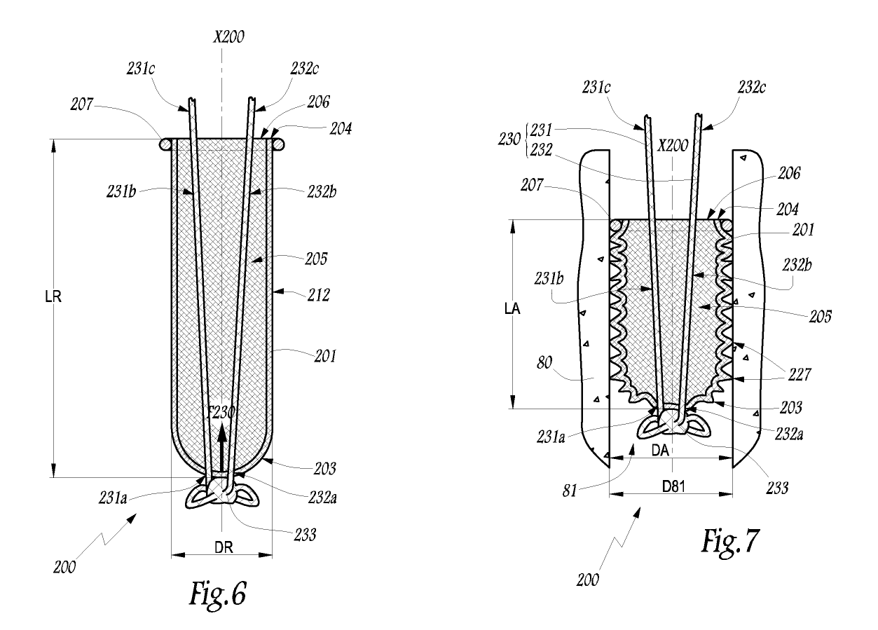

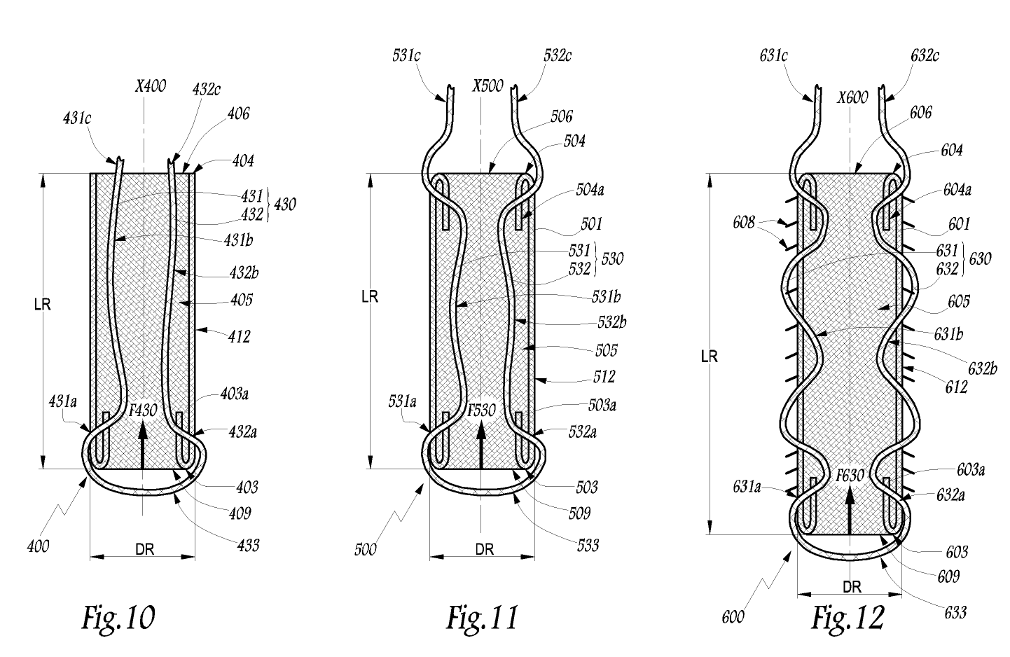

FIGS. 6 to 14 illustrate suture implant components 200, 300, 400, 500, 600, and 700. Such components may be implemented with a sleeve 50 and a rod 60, which are not shown in FIGS. 6 to 14 but which may operate in a similar fashion to that described with respect to FIGS. 1 to 5. Furthermore, the deformation of the components between the resting configuration and active configuration, as well as the anchoring of the components in the bone cavity 81 in their active configuration, are similar to the functioning of the component 100 as described above, according to embodiments of the present invention. By way of simplification, the bone 80 and the cavity 81 are not shown in FIGS. 10-14.

FIGS. 6 and 7 illustrate suture implant component 200. Certain elements of the component 200 are similar to elements of component 100 described above, and carry the same reference number increased by one hundred. This includes the axis X200, the body 201, the distal end 203, the proximal end 204, the opening 206, the interior space 205, the wall 212, the dimensions DR and LR in the resting configuration, the dimensions DA and LA in the active configuration, the bumps 227, the suture 230, as well as the strands 231 and 232 which include strand portions 231a, 232a, 231b, 232b, 231c and 232c.

A rigid ring 207 is attached to the proximal end 204 of the body 201 and reinforces the structure of the component 200, according to embodiments of the present invention. In practice, the dimensions of the ring 207 correspond to the dimensions of the cavity 81, such that the component 200 adheres to the wall of the cavity 81 as soon as it is inserted into the cavity 81, according to embodiments of the present invention. Moreover, the ring 207 is adapted to come into contact against the distal end 53 of the compression sleeve 50. As such, the risks of deformation or unforeseen sliding of the proximal end 204 out of the passage of the resting configuration to the active configuration are reduced.

The strands 231 and 232 traverse the distal end 203, respectively, at the level of portions 231a and 232a, and are connected by an arc 233 provided with a knot which is situated on the outside of the distal end 203 of the body 201, according to embodiments of the present invention. The portions 231b and 232b extend in the interior space 205 of the body 201 and across opening 206 without being interlaced with the wall 212, according to embodiments of the present invention. This implantation method for suture 230 in the component 200 is more rapid in comparison with the embodiment of FIGS. 1-5. Also, the strands 231 and 232 may be initially independent or connected, according to embodiments of the present invention.

The suture 230 cooperates with the body 201 of the component 200 via portions 231a and 232a and the arc 233 which is knotted. A tension F230 may be exerted on the distal end 203 of the body 201, in manipulating the suture 230, which may also deform the body 201 into an active configuration, according to embodiments of the present invention.

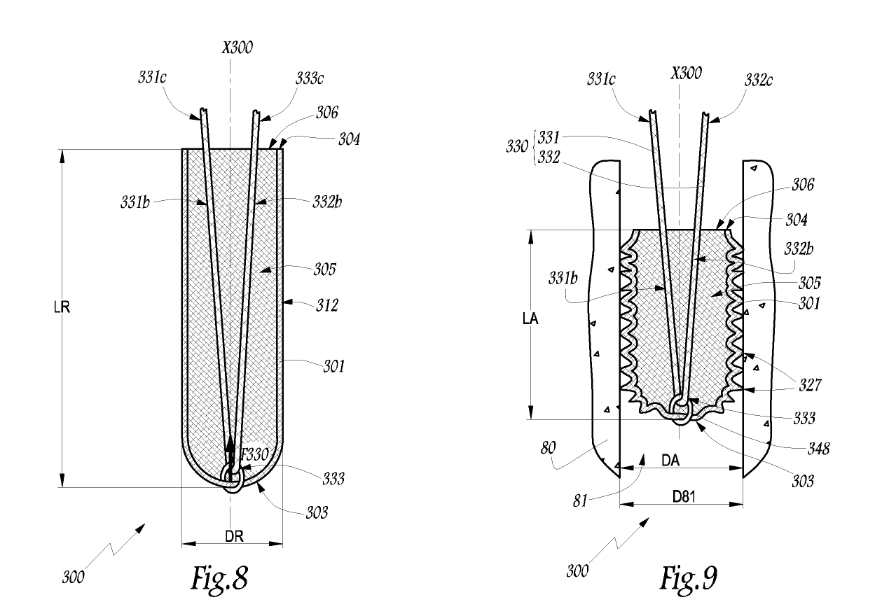

FIGS. 8 and 9 illustrate another suture implant component 300, according to embodiments of the present invention. Certain elements constituting the component 300 are similar to the elements of component 100 described above, and carry the same reference number increased by two hundred. This includes the axis X300, the body 301, the distal end 303, the proximal end 304, the opening 306, the interior space 305, the wall 312, the dimensions DR and LR in the resting configuration, the dimensions DA and LA in the active configuration, the bumps 327, the suture 330, as well as the strands 331 and 332 which are connected by an arc 333 and include suture strand portions 331b, 332b, 331c, and 332c, according to embodiments of the present invention.

The suture 330 does not traverse the body 301 at the level of the distal end 303 or along the length of the wall 312. Instead, a ring 348 is attached to the distal end 303. The ring 348 is adapted to receive the arc 333, according to a method for implanting the suture 330 in the component 300 which is more rapid in comparison with the embodiments of FIGS. 1-5. The ring 348 may be formed of a resorbable material, according to embodiments of the present invention.

The suture 330 cooperates with the body 301 of component 300 via the arc 333 and the ring 348. A tension F330 may be exerted on the distal end 303 of the body 301 in manipulating the suture 330, deforming the body 301 into the active configuration.

FIG. 10 illustrates a suture implant component 400 in a resting configuration, according to embodiments of the present invention. Certain elements constituting component 400 are similar to the elements of component 100 described above, and are given the same reference number increased by three hundred. This includes the axis X400, the body 401, the distal end 403, the proximal end 404, the opening 406, the interior space 405, the wall 412, the dimensions DR and LR of the resting configuration, the suture 430, as well as the strands 431 and 432 which are connected by an arc 433 and include strand portions 431a, 432a, 431b, 432b, 431c, and 432c.

The distal end 403 includes an opening 409, as well as a flap 403a which extends in the interior space 405 of the body 401 in the form of an annular hem. The strands 431 and 432 traverse not only the distal end 403 but also the wall 412, at the level of the flap 403a, then form the arc 433. As such, during the deformation of the body 401, the distal end 403 tends to contract in the direction of the axis X400.

Also, the suture 430 cooperates with the body 401 of the component 400 via portions 431a, 432a, and the arc 433. A tension F430 may be exerted on the distal end 403 of the body 401 in manipulating the suture 430, also deforming the body 401 into its active configuration, according to embodiments of the present invention.

FIG. 11 illustrates a suture implant component 500 in a resting configuration. Certain elements of component 500 are similar to elements of component 100 described above, and have the same reference number increased by four hundred. This includes the axis X500, the body 501, the distal end 503, the proximal end 504, the opening 506, the interior space 505, the wall 512, the dimensions DR and LR in the resting configuration, the suture 530, as well as the strands 531 and 532 which are connected by an arc 533 and include suture strand portions 531a, 532a, 531b, 532b, 531c, and 532c, according to embodiments of the present invention.

The distal end 503 includes an opening 509, as well as a flap 503a which extends in the interior space 505 of the body 501 in the form of an annular hem, according to embodiments of the present invention. Moreover, the proximal end 504 includes a flap 504a which extends in the interior space 505 of the body 501 in the form of an annular hem, according to embodiments of the present invention. The strands 531 and 532 traverse the wall 512, on one hand, at the level of the flap 503a to form the arc 533 and, on the other hand, at the level of the flap 504a to permit the manipulation of portions 531c and 532c. As such, during the deformation of the body 501, the distal end 503 and the proximal end 504 have a tendency to contract in the direction of the axis X500. In other words, interferences are formed in the intermediate portion between the ends 503 and 504, according to a localization that is easier to control in comparison with the component 400.

The suture 530 cooperates with the body 501 of the component 500 via the portions 531a, 532a, 531b, 532b, and the arc 533. A tension F530 may be exerted on the distal end 503 of the body 501 in manipulating the suture 530, thereby also deforming as well the body 501 to its active configuration, according to embodiments of the present invention.

FIG. 12 illustrates a suture implant component 600 in a resting configuration, according to embodiments of the present invention. Certain elements forming the component 600 are similar to elements of component 100, described above, and carrying the same reference number increased by five hundred. This includes the axis X600, the body 601, the distal end 603, the proximal end 604, the opening 606, the interior space 605, the wall 612, the dimensions DR and LR in the resting configuration, the suture 630, as well as strands 631 and 632 which are connected by an arc 633 and include suture strand portions 631a, 632a, 631b, 632b, 631c, and 632c, according to embodiments of the present invention.

The distal end 603 includes an opening 609, as well as a flap 603a which extends in the interior space 605 of the body 601 in the form of an annular hem. Moreover, the proximal end 604 includes a fold or flap 604a which extends in the interior space 605 of the body 601 in the form of an annular hem. The strands 631 and 632 traverse the wall 612, on one hand, at the level of the flap 603a to form the arc 633 and, on the other hand, at the level of the flap 604a to permit the manipulation of portions 631c and 632c, according to embodiments of the present invention.

During the deformation of the body 601, the distal end 603 and the proximal end 604 have a tendency to contract in the direction of the axis X600. The sides of the tubular wall 612 cooperate with the portions 631b and 632b of suture 630, for example the kind which deform the body 601 in an accordion-like fashion, by pleating and folding the body 601, according to embodiments of the present invention. The interferences formed on the wall 612 are separated in a homogeneous fashion between ends 603 and 604. The deformation of the component 600 is alogous to that of component 100, and is more easily controllable in comparison with components 400 and 500, according to embodiments of the present invention.

In addition, some monofilaments 608 may be mounted on the wall 612, on the exterior surface of the body 601, according to embodiments of the present invention. The monofilaments 608, or other more rigid fibers, may be woven into the body 601, according to a particular pattern, such that while the body 601 is deformed, the monofilaments 608 act as barbs and reinforce the adherence of the component 600 in the cavity 81.

According to one variation, the monofilaments 608 may be disposed on a portion of the length LR of the body 601, according to the desired geometry in the active configuration, according to embodiments of the present invention.

The suture 630 cooperates with the body 601 of the component 600 via the portions 631, 632a, 631b, 632b, and the arc 633. A tension F630 may be exerted on the distal end 603 of the body 601 in manipulating the suture 630, deforming the body 601 into its active configuration.

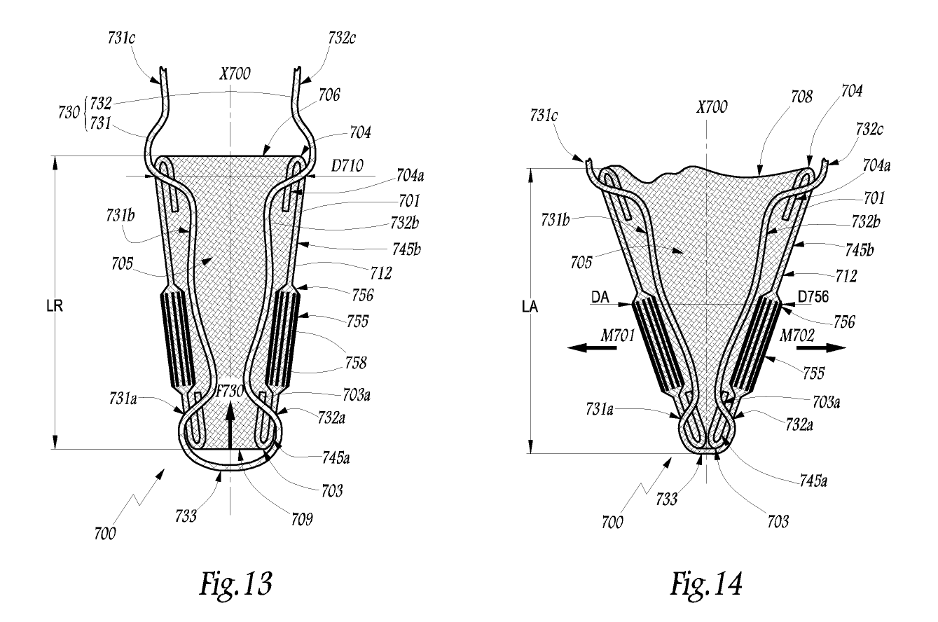

FIGS. 13 and 14 illustrate a suture implant component 700, according to embodiments of the present invention. Certain elements of component 700 are similar to those of component 100, described above, and carrying the same reference number increased by six hundred. This includes the axis X700, the body 701, the distal end 703, the proximal end 704, the opening 706, the interior space 705, the wall 712, the dimensions DR and LR in the resting configuration, the length LA in the active configuration, the suture 730, as well as the strands 731 and 732 which are connected by an arc 733 and include suture strand portions 731a, 732a, 731b, 732b, 731c, and 732c, according to embodiments of the present invention.

The component 700 is illustrated, on one hand, in a resting configuration in FIG. 13 and, on the other hand, in a “semi-active” configuration in FIG. 14, in which the body 701 is illustrated as only partially deformed with respect to its active configuration. The body 701 includes a wall 712 which includes a portion 745a at a side of the distal end 703, an intermediate portion 755 and a portion 745b at a side of the proximal end 704, according to embodiments of the present invention. In the semi-active configuration of FIG. 14, the portions of the wall 745a and 755 are experiencing deformation. In the active configuration, the portion of the wall 745b is crushed against the wall of the bone cavity 81 and undergoes an accordion-type deformation analogous to those deformation modes described above, according to embodiments of the present invention.

In addition, monofilaments 758 are positioned in the portion of wall 755, for example woven in the interior of the body 701, according to embodiments of the present invention. Such monofilaments 758 are more rigid fibers than the fibers constituting the body 701, such that, during the deformation of the body 701, the portion 755 presents a superior resistance to deformation than portions 745a and 745b, according to embodiments of the present invention.

The distal end 703 includes an opening 709, as well as a flap 703a which extends in an interior space 705 of the body 701, against the portion of the wall 745a, in the form of an annular hem, according to embodiments of the present invention. In addition, the proximal end 704 includes a flap 704a which extends in the interior space 705 of the body 701, against the portion 745a, in the form of an annular hem. The strands 731 and 732 traverse the wall 712, on one hand, at the level of the flap 703a to form the arc 733 and, on the other hand, at the level of the flap 704a to permit manipulation of the portions 731c and 732c, according to embodiments of the present invention.

During the deformation of the body 701, the distal end 703 and the proximal end 704 have a tendency to contract in the direction of the axis X700. The portion 745b deforms in accordion style, while the portions 745a and 755 deform in umbrella style. In fact, the portion 745a contracts and the distal opening 709 closes, while the portion 755 opens radially in a manner comparable to an umbrella, according to a displacement which is represented by arrows M701 and M702 in FIG. 14, in the semi-active configuration, according to embodiments of the present invention. Also, the pressure of the portion 755 against the bone cavity 81 is experienced at the perimeter 756, which has a diameter D756. In active configuration, the diameter D756 is greater than the diameter DR and corresponds to diameter DA, which is itself equal to the diameter D81 of the cavity 82, according to embodiments of the present invention.

In practice, the component 700 adheres to the cavity 81, on one hand, at the level of the perimeter 756 and, on the other hand, at the level of the portion 745b of the wall 712 which has been deformed in accordion fashion. This accordion-like deformation of the portion 745b around the axis X700 forms several folds. The suture 730 cooperates with the body 701 of the component 700 via portions 731a, 732a, 731b, 732b, and the arc 733. A tension F730 may be exerted on the distal end 703 of the body 701 in manipulating the suture 730, in order to deform the body 701 into the active configuration, according to embodiments of the present invention.

In one variation, the monofilaments 758 may be disposed on different portions of the wall 745, for example distributed along the length LR of the body 701 according to the desired geometry of the active configuration. The monofilaments 758 may be arranged in an annular fashion, arranged axially, or both. For example, two end portions of the wall 745 may include monofilaments 758, while the intermediate portion does not include monofilaments 758, according to embodiments of the present invention. In this case, the body 701 deforms in a manner similar to a “double umbrella.” The supplemental reinforcements may be integrated into the body 701 to form the “ribs” of the umbrella, according to embodiments of the present invention.

The opening which is configured to receive the suture is located on the side of the proximal end of the body, such that the suture penetrates into the interior space of the body and cooperates with the body at least on the distal end side, according to embodiments of the present invention.

However, according to other embodiments of the present invention, the suture may be connected solely to the distal end of the body, or may even be bent across the wall of the body, or may even be directly woven into the body. The multiple passages of the suture to the inside and the outside of the walls may enhance the bending, in other words the accordion-like deformation, according to embodiments of the present invention. The body of the component may include an open or closed distal end, according to embodiments of the present invention.

According to one alternative embodiment, the suture implant component does not have a tubular body, but instead has the form of a slender cone. While the conical wall is reduced on the side of the distal end and a tension is exerted on the distal end, the distal end is pulled across the interior space of the body and the conical wall is forced to extend transversally in the bone cavity. In other words, while the component is woven or braided with a conical form, its geometry in an active configuration may be more easily controlled.

According to another embodiment of the present invention, the implant component includes a cinch-type mechanism. Such mechanism may be formed by a material similar to a suture strand, without metal, and integrated in the body of the implant component during its fabrication, for example by weaving. One cinch mechanism may extend from the distal end of the body, while the suture strand may be wrapped around the proximal end of the body, then traverse the cinch mechanism. Thus, the suture strand is locked in the component, with its free ends available for the formation of a connecting knot by the surgeon.

According to another embodiment of the present invention, a clip, a staple, a ring, or a button may be disposed in the component such that the deformed body may be locked in the active configuration.

In addition, the point of the insertion rod of the component in the bone cavity may be configured for punching or piercing. Also, the surgeon may not need to pre-pierce a bone cavity in the bone, as the point of the rod may be adapted to directly form the bone cavity, according to embodiments of the present invention. The component may include a distal opening so that the point of the rod does not damage the body of the component, according to embodiments of the present invention.

According to another alternative embodiment, the suture implant mechanism which is available to the surgeon does not include an insertion rod and/or a compression sleeve. In such case, other mechanisms for manipulation and insertion of the suture implant component in the bone cavity may be put to use. For example, the insertion mechanism may be removable and may be withdrawn from the component in the active configuration, according to embodiments of the present invention.

According to yet another alternative embodiment, the component includes a polymer, a cement, or an inflatable balloon which is introduced into the body for extending the diameter of the body until the body reaches the diameter of the bone cavity. Also, the component may be anchored against the cavity by the action of an internal pressure resulting from the deformation of the body. The expansion element may remain with the implant or be withdrawn after expansion. The expansion element may also be localized to promote a particular geometry in the active configuration, such as a particular geometry corresponding to that of an umbrella, according to embodiments of the present invention.

No matter what the mode of operation of the suture implant component according to embodiments of the present invention, the wall of the body of the implant is deformable, for example, in the manner of an accordion, along its longitudinal axis. In this way, several folds are formed on at least a portion of the wall, around the longitudinal axis.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What is claimed is:

1. A system for suture implantation, the system comprising:
a suture implant component, comprising:
a body which extends along a longitudinal dimension, the body comprising
an opening configured to receive a suture,
the body configured for cooperation with the suture,
the body being deformable, upon application of a tension to the suture, between a resting configuration in which the body has a first longitudinal dimension and a first transverse dimension, and an active configuration in which the body has a second longitudinal dimension smaller than the first longitudinal dimension and a second transverse dimension larger than the first transverse dimension; and
a wall arranged about the longitudinal dimension, wherein the wall is deformable in an accordion-like manner as the body deforms from the resting configuration to the active configuration.

2. The system of claim 1, further comprising:
the suture, wherein the suture cooperates with the body to form a general U or V shape, and wherein the suture engages the body at least a distal end of the body.

3. The system of claim 1, wherein the wall is deformable in the accordion-like manner by pleating, bending, crushing, or contraction as the body deforms from the resting configuration to the active configuration.

4. The system of claim 1, wherein the wall is tubular or conical.

5. The system of claim 1, wherein the body is formed about a longitudinal axis extending along the longitudinal dimension, wherein the wall is arranged about the longitudinal axis, and wherein in the active configuration, the wall forms several folds about the longitudinal axis.

6. The system of claim 1, wherein the body is woven or braided with biocompatible fibers formed with one or a combination of materials selected from the group consisting of: polyester, polyethylene, and polyhydroxyalkanoate.

7. The system of claim 6, wherein the biocompatible fibers are resorbable fibers.

8. The system of claim 1, wherein the suture implant component comprises at least two types of fibers each having a different diameter.

9. The system of claim 2, wherein the suture comprises a first strand and a second strand, wherein the first and second strands traverse the distal end of the body and are connected by an arc, wherein the arc comprises a knot which is located outside of the distal end of the body.

10. The system of claim 1, wherein monofilaments are distributed on an outside of the wall along at least a portion of the longitudinal dimension.

11. The system of claim 1, wherein monofilaments are integrated into the body along at least a portion of the longitudinal dimension.

12. The system of claim 1, wherein the body further comprises a reinforcing ring located at a proximal end of the body.

13. The system of claim 1, wherein a proximal end of the body comprises an opening while a distal end of the body is closed.

14. The system of claim 1, further comprising a locking element configured to maintain the body in the active configuration.

15. The system of claim 14, wherein the locking element is a clip, a staple, a ring, or a button disposed in the body.

16. The system of claim 2, wherein the body further comprises a cinch mechanism at its distal end, wherein the cinch mechanism is configured to receive and lock the suture, the cinch mechanism formed of the same material as the suture and woven into the body.

17. The system of claim 2, further comprising a means for attaching the suture to the distal end of the body.

18. The system of claim 1, further comprising:
an insertion rod; and
a tubular sleeve.

19. The system of claim 1, further comprising:
a rod adapted to maintain the first longitudinal dimension and the first transverse dimension of the body in the resting configuration, the rod configured to extend through the opening and to occupy an interior space of the body; and
a sleeve, wherein the sleeve is tubular and adapted to maintain a proximal end of the body in position during deformation of the body from the resting configuration to the active configuration, by pressing against the proximal end of the body.

20. The system of claim 19, wherein the rod comprises a point, wherein the point is configured to punch or pierce a hole into bone for insertion of the suture implant component into the bone.

21. A method for implanting a suture implant component, wherein the suture implant component comprises a body which extends along a longitudinal axis, the body comprising an opening configured to receive a suture, the body configured for attachment to the suture, the body further comprising a wall arranged about the longitudinal axis, the method comprising:
inserting a rod into the body;
inserting a sleeve over the rod, into sliding engagement with the rod;
inserting the body and a distal end of the rod into a bone cavity;
removing the rod from the body;
maintaining the sleeve in place against a proximal end of the body as a tension force is applied to the suture to deform the body, in an accordion-like manner, from
a resting configuration in which the body has a first longitudinal dimension and a first transverse dimension, to
an active configuration in which the body has a second longitudinal dimension smaller than the first longitudinal dimension and a second transverse dimension larger than the first transverse dimension, thereby securing the body within the bone cavity.
